(19) United States
(12) Patent Application Publication
(10) Pub. No.: US 2023/0381313 A1
(43) Pub. Date: Nov. 30, 2023

(54) CD16high CD57high NK-92MI Cells

(71) Applicant: ImmunityBio, Inc., Culver City, CA (US)

(72) Inventors: Scotty Cadet, Culver City, CA (US); Syed Raza Ali, Culver City, CA (US); Manju Saxena, Culver City, CA (US); Barry J. Simon, Culver City, CA (US)

(73) Assignee: ImmunityBio, Inc., Culver City, CA (US)

(21) Appl. No.: 18/195,588

(22) Filed: May 10, 2023

Related U.S. Application Data
(60) Provisional application No. 63/345,275, filed on May 24, 2022.

(51) Int. Cl. A61K 39/00; C12N 5/0783; C07K 14/735; C07K 14/705; A61P 35/00

(52) U.S. Cl. CPC A61K 39/4613; C12N 5/0646; C07K 14/70535; C07K 14/70596; A61P 35/00

(57) ABSTRACT

Cells and cell-based therapeutic compositions and methods are presented in which the cells are CD16+CD57+NK-92MI cells that natively express CD16 and CD57 and that exhibit IL-2 independent growth.

This application claims priority to our copending U.S. provisional patent application with the Ser. No. 63/345,275, which was filed May 24, 2022, and which is incorporated by reference herein.

### Field of the Invention

The field of the invention is composition and methods for cell based therapeutics, and especially as it relates to NK cells with enhanced cytotoxicity that natively express CD16 and CD57.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Natural killer (NK) cells constitute a group of innate immune cells, which are often characterized as cytotoxic lymphocytes that exhibit antibody dependent cellular toxicity via target-directed release of granulysin and perforin. While more recently NK cells have become a significant component of certain cancer treatments, generation of therapeutically meaningful quantities of NK cells, and especially autologous NK cells, remained a significant obstacle as the fraction of NK cells in whole blood is relatively low.

To obtain therapeutically meaningful quantities of NK and NK-like cells, NK cells can be generated from various precursor cells. For example, various stem cell factors (SCF), FLT3 ligand, interleukin (IL)-2, IL-7 and IL-15 have been reported in various in vitro approaches to induce and expand cord blood-derived cytokine-induced killer (CIK) cells (Anticancer Research 30: 3493-3500 (2010)). Similarly, CD34⁺ hematopoietic cells can be exposed to IL-12 and other agents as is reported in US 2018/0044636. In still other approaches, human hemangioblasts were sequentially exposed to two different cytokine cocktails as described in WO2011/068896, and different cytokine cocktails were used with post-embryonic hematopoietic stem cells as taught in WO2012/128622. While at least some of these methods provide a significant n-fold expansion of NK cells, methods and reagents for such expansion are both time and resource demanding. Still further, it should be noted that many of the known methods also require NK cell culture on a feeder cell layer, which is often problematic from a technical and a regulatory perspective.

To avoid issues associated with generation of therapeutic quantities of NK cells from precursor cells or isolated NK cells, an NK-92 cell line can be employed. NK-92 cells are interleukin-2 (IL-2) dependent natural killer cells derived from peripheral blood mononuclear cells from a 50-year-old, white male with rapidly progressive non-Hodgkin's lymphoma, and are commercially available as CRL-2407 from ATCC. Advantageously, NK-92 cells can be grown to large scale, and the ability of the natural killer cell line NK-92 to kill efficiently a broad range of malignant cells makes it suitable for cellular immunotherapy of cancer. However, cells of the NK-92 cell line generally do not express CD16 and as such lack the capability to bind to a targeting antibody for target specific cytotoxicity (and especially antibody-dependent cellular cytotoxicity (ADCC)).

More recently, NK cells were produced that expressed CD16 from a recombinant nucleic acid that was transfected into the NK cells as is described, for example, in US 2019/0321402. Unfortunately, such RNA-based expression will typically not be stable over multiple generations during NK cell expansion to obtain significant quantities of NK cells for treatment. In an alternate approach, a FACS sorting strategy was used on the NK-92 cell line to select NK cells with endogenous CD16 expression as is described in WO 2020/1504752018 (see also: Zih-Fei Cheng (2021) Biochemistry and Biophysics Reports Volume 26). Here, a human NK-92-derived cell line, oNK-1, was established that endogenously expressed CD16, along with high levels of NK activation markers and low levels of NK inhibitory markers characteristic for the parental NK-92 cell line.

Interestingly, when these cells were covalently conjugated with a monoclonal antibody, Trastuzumab, the so modified cells exerted human epidermal growth factor 2 (HER2) binding specificity and enhanced cytotoxicity against various types of cancer cells with grade 1 (MCF-7), grade 2 (OVCAR-3) and grade 3 (SK-OV-3) HER2 expression compared to control oNK cells in vitro (see e.g., Proceedings of the Annual Meeting of the American Association for Cancer Research 2020;2020 Apr. 27-28 and Jun. 22-24. Philadelphia (PA): AACR; Cancer Res 2020;80(16 Suppl):Abstract nr 2169). Unfortunately, production and CD16 expression in oNK cells required very high amounts of IL-2 (500 unit/mL).

NK-92MI (ATCC: CRL-2408) is an IL-2-independent variant of NK-92 that was made using particle-mediated gene transfer of the hIL-2 cDNA. NK-92MI cells show potent cytotoxicity against different tumor cells. Nonetheless, the Fc-receptor (CD16) that usually mediates ADCC is altogether missing or present only at very low level on NK-92MI cells, and these cells will as such fail to induce ADCC for target specific cell killing. While it has been shown that CD16-mediated ADCC activity is more potent if circulating NK cells are also positive for CD57 (see e.g., Sandra Lopez-Vergès (2010) Blood 2010 116(19)), the fraction of CD16+ CD57+ cells in blood is very low as these cells represent mature NK cells. Consequently, enrichment and propagation into therapeutically meaningful quantities has not been achieved. NK-92MI chimeric cells were also generated to express recombinant chimeric receptors that can bind the Fc portion of human immunoglobulins using viral transduction (see e.g., Ying Chen (2017) Oncotarget 6;8(23)). However, CD57 expression of these cells was neither reported nor reasonably expected.

Thus, even though various compositions and methods of NK cell therapy are known in the art, all or almost all of them suffer from several drawbacks, particularly where such cells are required in significant quantities in an economic manner and where such cells express CD16 and CD57 for enhanced cytotoxicity.

### SUMMARY OF THE INVENTION

The inventive subject matter is directed to various cells and cell-based compositions and methods therefor in which the cells are CD16+CD57+NK-92MI cells that natively express CD16 and CD57 and that exhibit IL-2 independent growth. Most notably, such cells had significantly enhanced cytotoxicity and could be produced in a simple and cost effective manner.

In one aspect of the inventive subject matter, the inventors contemplate a CD16+CD57+NK-92MI cell that natively expresses CD16 and CD57 and that exhibits IL-2 independent growth. Most typically, the CD16 is CD16a or CD16b. Especially contemplated cells express NKG2D in higher quantities as compared to the parental NK-92MI cells (ATCC: CRL-2408). Moreover, CD3 is typically not present on a surface of the cell whereas CD56 is typically present on the surface of the cell. Moreover, the cells presented herein may further include one or more of CD4, CD25, NKp30, NKp44, NKp46, CD27, OX40, CD107a, NKG2A, PD-1, TIGIT on the surface of the cell.

Phenotypically, the cells presented herein will generally have enhanced direct cytotoxicity against Ramos cells as compared to the parental NK-92MI cells (ATCC: CRL-2408), and/or exhibits strong ADCC in the presence of a target cell and an antibody against a surface protein on the target cell. Moreover, contemplated cells will also have, post-thaw and expansion, high levels of expression of CD57, CD16, and NKG2D as compared to the parental NK-92MI cells (ATCC: CRL-2408). Still further, in at least some embodiments the cells will have a faster replenishment post degranulation, and/or a faster cell doubling time as compared to the parental NK-92MI cells (ATCC: CRL-2408).

Where desired, the CD16+CD57+NK-92MI cells may also be transfected with a recombinant nucleic acid that encodes a chimeric antigen receptor, a homing receptor, a chemokine receptor, a TGF-β trap, and/or a checkpoint inhibitor. Alternatively, or additionally, the CD16+CD57+NK-92MI cells may also have an antibody or antibody fragment bound to the surface of the cell (e.g., via CD16 or a linker).

In another aspect of the inventive subject matter, the inventors also contemplate a composition that comprises a plurality of NK-92MI cells as presented herein in a medium, wherein at least 10%, or at least 50%, or at least 90% of the NK-92MI cells are CD16+CD57+NK-92MI cells that natively express CD16 and CD57 and that exhibit IL-2 independent growth. As will be appreciated, contemplated media include a growth medium, a cryopreservation medium, and a pharmaceutically acceptable medium for infusion. Moreover, the composition may further comprise an antibody, which may or may not be bound to the CD16 of the CD16+CD57+NK-92MI cell. In further contemplated aspects, the plurality of cells is between 1×10⁸ to 1×10¹⁰ cells, the medium is a pharmaceutically acceptable medium for infusion, and the cells and the medium are disposed in a polymeric bag.

In a further aspect of the inventive subject matter, the inventors contemplate cell culture comprising a plurality of dividing cells in a culture medium, wherein the cells are CD16+CD57+NK-92MI cells as presented herein wherein the medium is substantially free of IL-2. In typical embodiments, the plurality of dividing cells are maintained in a single culture container, preferably from the start of the culture and during growth until a predetermined quantity of cells is obtained. Therefore, contemplated cell cultures may contain at least 1×10⁷ cells per culture container. Most typically, but not necessarily, the medium contains human AB serum or is a serum-free medium.

In still a further aspect of the inventive subject matter, the inventors contemplate a method of preparing CD16+CD57+NK-92MI cells that natively express CD16 and CD57 and that exhibit IL-2 independent growth. Such method typically includes a step of providing a plurality of NK-92MI cells, and another step of using anti-CD16 antibodies and anti-CD57 antibodies to enrich the CD16+CD57+NK-92MI cells that natively express CD16 and CD57.

For example, each of the anti-CD16 antibodies and anti-CD57 antibodies may be fluorescence-labeled, and the antibodies may be used for (typically sequential) enrichment sing fluorescence activated cell sorting (FACS). As will be readily appreciated, the FACS may be performed using single pass sorting or using iterative rounds of FACS. Moreover, contemplated methods may also include a step of expanding the enriched population of the CD16+CD57+NK-92MI cells.

In yet another aspect of the inventive subject matter, the inventors contemplate a method of treating a cancer that includes a step of administering to an individual in need thereof a therapeutically effective quantity of cells as presented herein or a composition comprising such as cells. Most typically, the individual is a mammal (e.g., human), and/or the cancer is a solid cancer. As will be readily appreciated, the cells or the composition is administered by infusion, and the treatment may also include a step of co-administering an antibody, a checkpoint inhibitor, an immune stimulant, and/or a cancer vaccine.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts exemplary FACS results for enrichment of CD16 and CD57 in sorted NK-92MI cells.

FIG. 2 shows exemplary results for surface phenotyping of parental and sorted NK-92MI cells.

FIG. 3 depicts graphs showing exemplary results for ADCC of sorted NK-92MI cells against Ramos target cells in the presence of Rituxan antibody.

FIG. 4 depicts a graph showing exemplary results for ADCC of sorted NK-92MI cells against HCC827 target cells in the presence of Avelumab antibody.

FIG. 5 depicts exemplary FACS results establishing that NK-92MI sorted cells maintained high expression of CD57 and CD16 after freeze-thaw.

FIG. 6 shows exemplary results for surface phenotyping of parental and sorted NK-92MI cells after freeze-thaw.

FIG. 7 depicts graphs showing exemplary results for ADCC of sorted NK-92MI cells against Ramos target cells after freeze-thaw.

### DETAILED DESCRIPTION

The inventors have discovered that NK cells with high general and target-specific cytotoxicity can be prepared that not only express CD16 and CD57 in high quantities, but that can also be grown without the need for exogenously added IL-2. Unexpectedly, these cells were isolated from an NK-92MI cell line that was previously not known to express CD16 and CD57 in significant quantities. In this context, it should be appreciated that the NK-92MI cell line is are not a collection of naturally occurring cells, but a cell line that was established by transformation of an NK-92 cell line with human IL-2 (hIL-2) cDNA by particle-mediated gene transfer (see Hum Gene Ther. 1999 May 20;10(8):1359-73).

While not wishing to be bound by a specific theory or hypothesis, the inventors contemplate that upon expansion of an NK-92MI cell line, some of the cells undergo certain genetic changes, and possibly epigenetic changes, that can lead to native and stable expression of CD16 as well as CD57. While rare in the overall population of cells in the NK-92MI cell line, the inventors noted that such cells can be enriched via affinity based separation methods. Notably, such enrichment was facilitated by the fact that NK-92MI cells express from a recombinant and genomically integrated nucleic acid sequence IL-2, rendering such cells independent from exogenously added IL-2.

In further unexpected findings, the inventors also observed that the so enriched CD16+CD57+NK-92MI cells also expressed NKG2D, a master regulator of cytotoxicity, in significantly higher quantities as compared to the NKG2D expression of the NK-92MI cell line. Such increased presence of NKG2D even further enhanced general and ADCC-type cytotoxicity of the isolated/enriched CD16+CD57+NK-92MI IL-2 independent cells.

As used herein, the phrase “cells that natively express CD16 and CD57” or “native and stable expression of CD16 and CD57” refers to cells that express CD16 and CD57 from respective genes in the genome of the cell, wherein these genes are not recombinant genes introduced into the cell. As also used herein, the phrase “exhibits IL-2 independent growth” with respect to a cell or cells refers to the cells' capability to undergo n-fold expansion (with n being at least 10) in a culture medium that, as supplied, is free of IL-2, to which no IL-2 was exogenously added, or to which no IL-2 was provided by a feeder cell layer. Similarly, as still further used herein, the phrase “medium is substantially free of IL-2” refers to a medium that has insufficient quantities of IL-2 to support growth of NK-92 cells (ATCC: CRL-2407). Therefore, a medium that is substantially free of IL-2 will contain IL-2 in a quantity of less than 10 IU/mL, or less than 5 IU/mL, or less than 1 IU/mL. However, it should be noted that the CD16+CD57+NK-92MI cells may secrete IL-2 into the medium, and all secreted quantities are included in the scope of this definition.

While the CD16+CD57+NK-92MI IL-2 independent cells were isolated from the NK-92MI cell line, it should be appreciated that numerous other cell lines, including NK-92 and genetically modified NK-92 cells lines (e.g., NK-92CI) are also deemed suitable as a parental cell line. Most typically, where the NK-92 cell line is genetically modified, it is generally preferred that such modification is a genetically stable modification as that the modification is passed down the progeny cells. Therefore, especially contemplated genetic modifications include artificial chromosomes, viral genome insertions into the cell genome, CRISPR-based modifications, etc. In some embodiments, the genetic modification will include addition of a nucleic acid that encodes CD16 and/or CD57, whereas in other modifications the genetic modification will include addition of a nucleic acid that encodes IL-2 (which may or may not include a cytoplasmic or endoplasmic reticulum retention sequence). Further contemplated genetic modification will include addition of a nucleic acid that encodes one or more recombinant proteins to enhance cytotoxicity, to target the cell to a specific location, etc., and especially contemplated recombinant proteins include a chimeric antigen receptor, a homing receptor, a chemokine receptor, a (secreted or membrane bound) TGF-β trap, and/or a checkpoint inhibitor.

In further contemplated aspects, it should be noted that the parental cell line (e.g., NK-92MI) may be used for cell sorting or other type of enrichment for CD16 and CD57 directly after thawing from a cryopreservation medium, or after passage of at least 1, or at least 2, or at least 3, or at least 5, or at least 10, or at least 20 generations in a culture medium. Most typically, such culture medium will be serum free and will not contain exogenous IL-2. In other embodiments, the medium contains human AB serum but no exogenously added IL-2. Likewise, it is generally preferred that the culture medium will not include additional feeder cell layers.

For cell isolation and enrichment, it is generally preferred that the cells are isolated using a selection process that includes use of anti-CD16a antibodies and anti-CD57 antibodies. However, in alternative embodiments, the antibodies may also be anti-CD16b antibodies, anti-CD16c antibodies, anti-CD32 antibodies, and/or anti-CD64 antibodies. Most commonly, the cell selection process will be a FACS process, however, various alternative selection processes are also deemed suitable for use herein and include magnetic bead separation in which the antibodies are coupled to the beads, microwell plate separation where the antibodies are coupled to the walls of the wells, microfluidic separation where the antibodies are coupled to a wall of a microfluidic channel, etc. Regardless of the manner of separation, it should be appreciated that the separation of the cells can be performed in a single pass using both the anti-CD16a antibodies and the anti-CD57 antibodies, or in multiple sequential passes where the cells are first enriched in CD16+cells that are then further enriched for CD57+cells.

As will also be readily appreciated, the cells may be subjected to a growth/expansion step, especially where the isolation is performed in a sequential fashion. Such growth and expansion may be started from a number of cells (e.g., raw output of an isolation step) or from a reduced number of cells. Such reduced number may be obtained by sub-cultivation under selected conditions, or by serial dilution to ultimately produce a monoclonal cell product. Once the desired CD16+CD57+NK-92MI IL-2 independent cells are obtained, it is contemplated that these cells can then be further characterized for one or more parameters and that the cells can be confirmed as being positive for CD16 and CD57.

Where the CD16+CD57+NK-92MI IL-2 independent cells are not of monoclonal origin, it is also contemplated that the so obtained cell population can be further tested for and/or selected for additional desirable traits. Among other things, the CD16+CD57+NK-92MIL-2 independent cells can be selected or confirmed for rapid replenishment post-degranulation, decreased cell doubling time, optimal growth density, etc. (all relative to the parental NK-92MI cell line). Similarly, the CD16+CD57+NK-92MI IL-2 independent cells can be selected or confirmed for cytotoxicity/potency against a variety of cancer cells (which may be cells of solid cancers or blood borne cancers), cytokine expression and especially IFN-γ secretion, migration to a target tissue, etc.

Once isolated, it should be appreciated that the cells can be further genetically modified to include one or more desirable properties, typically by expression of one or more recombinant proteins. For example, especially preferred additional proteins suitable for expression include a chimeric antigen receptor preferably targeting a cancer or tumor associated antigen (e.g., CD19, CD20, GD2, HER-2, CD30, EGFR, FAP, CD33, CD123, PD-L1, IGF1R, CSPG4, or B7-H4) or neoantigen, a homing receptor (e.g., G protein-coupled receptor (GPCR), a chemokine receptor such as CCR7, CXCR2, or the receptor for CXCL14, a cytokine receptor, a cell adhesion molecule, a selectin, or an integrin), a TGF-β trap that may be membrane bound or secreted, and/or a checkpoint inhibitor. Exemplary proteins, sequences, and constructs for such modifications are described in WO 2020/028656, incorporated by reference herein. Additionally, it should be appreciated that the cells presented herein may be further modified with one or more target specific binders such as antibodies and fragments thereof, including scFv constructs.

Consequently, it should be appreciated that the cells presented herein can be used in a variety of therapeutic uses, and especially for infusion to an individual in need thereof. Thus, the inventors also contemplate composition in which the cells presented herein are placed in a growth medium for preparation of therapeutic quantities of cells, in a cryopreservation medium for storage of such cells, or in a pharmaceutically acceptable medium for infusion.

As will be readily appreciated, the cells presented herein may be administered to a subject by a variety of routes, and an especially preferred route is intravenous infusion over a period of time. Typically, for a single dose of the cells, the period of time is between 5 and 130 minutes. Optionally, the period of time is between 90 and 120 minutes. Optionally, the period of time is between 15 to 30 minutes. Preferably, the cells presented herein can be administered daily, twice or thrice weekly, once weekly, twice weekly, once monthly, or in longer intervals.

With respect to the number of cells administered, it is contemplated that the cells presented herein can be administered to the subject from about 1000 cells/infusion to up to about 10 billion cells/infusion, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ cells per infusion, or any ranges between any two of the numbers, end points inclusive. For example, from 1×10⁸ to 1×10¹⁰ cells are administered to the subject. As will be readily appreciated, the cells can be administered from a production process without intermediate storage in a cryopreservation medium, or the cells can be administered after thawing. Where desired, the cryopreservation medium may be replaced with an infusion medium prior to infusion. In further contemplated aspects, it should be appreciated that the cells presented herein may also be irradiated to reduce or abrogate proliferation, especially where the cells are administered in a therapeutic composition. Accordingly, all manners or irradiation are deemed suitable for use herein and particularly include low-energy e-beam irradiation and gamma irradiation, typically at dosages between 2 and 20 Gy, and more typically between 6 and 10 Gy (see e.g., Front. Immunol. June 2021, Vol. 12, Article 684052).

Accordingly, it should be appreciated that the CD16+CD57+NK-92MI cells of the inventive subject matter can be used in a variety of therapeutic needs. However, it is especially contemplated that the cells are used in a method of treating cancer (e.g., solid tumor or blood borne cancer) in a subject where a therapeutically effective quantity of cells are administered, typically in pharmaceutically acceptable composition that includes such cells. Advantageously, these cells will form part of a multimodal treatment strategy that includes additional therapeutic agents such as an antibody, a checkpoint inhibitor, an immune stimulant, a cancer vaccine, and/or metronomic low-dose chemotherapy.

### Examples

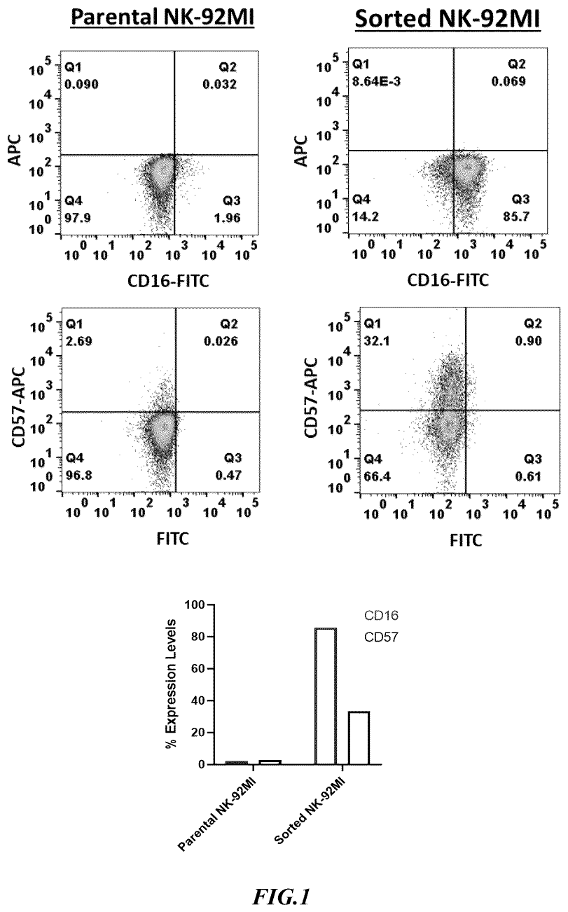

The inventors sorted CD57+CD16+NK cells from a parental NK-92MI cell line (e.g., commercially available from ATCC as CRL-2408) using BD FACS Melody cell sorter and fluorescence labeled anti-CD16 and anti-CD57 antibodies. Post sorting, the cells were cultured up to 126 days and high CD16 and CD57 expression was observed by flow cytometry (1.98% CD16 in parental cell vs 85.7% CD16 in sorted cells and 2.69% CD57 in parental cell vs 32.1% CD57 in sorted cells). FIG. 1 depicts exemplary results for such enrichment of CD16 and CD57 in sorted NK-92MI cells. Here, the surface marker expression on parental (unsorted) and sorted NK-92MI cells was analyzed by flow cytometry-based staining, and the results are presented as dot plots. The bar graph compares the percent expression of respective markers on parental and sorted cells.

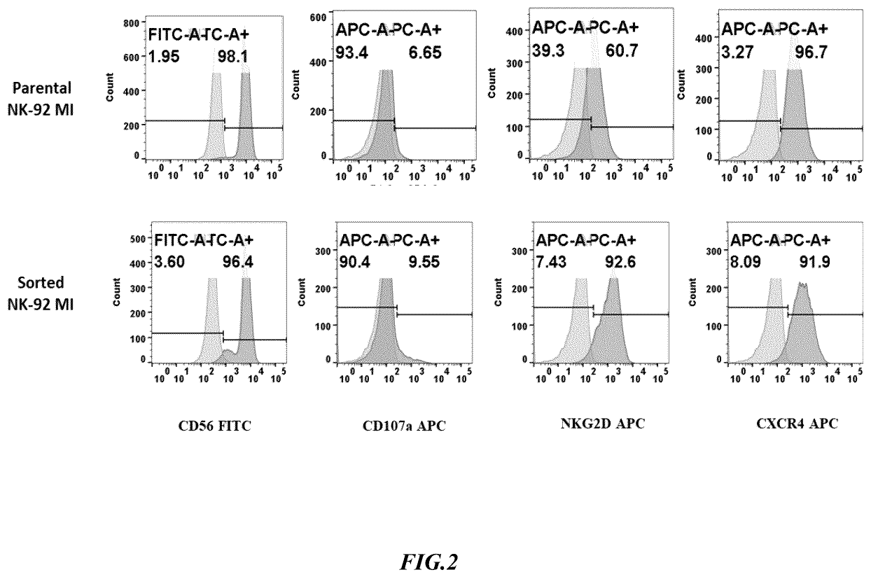

Unexpectedly, the sorted cells also displayed higher expression of NKG2D, a master regulator of cytotoxicity, when compared to the parental cells as can be taken from FIG. 2. On the other hand, expression of receptors such as CD56 and CXCR4 were comparable on both cell lines as can also be seen from FIG. 2. Notably, the sorted cells also maintained high viability throughout the culturing period. FIG. 2 depicts exemplary results for surface phenotyping of parental NK-92MI and sorted CD16+CD57+NK-92MI cells. Here, surface marker expression was analyzed by flow cytometry-based staining and the results are presented below as histogram. The light grey and dark grey indicate the marker staining on unstained and stained cells, respectively.

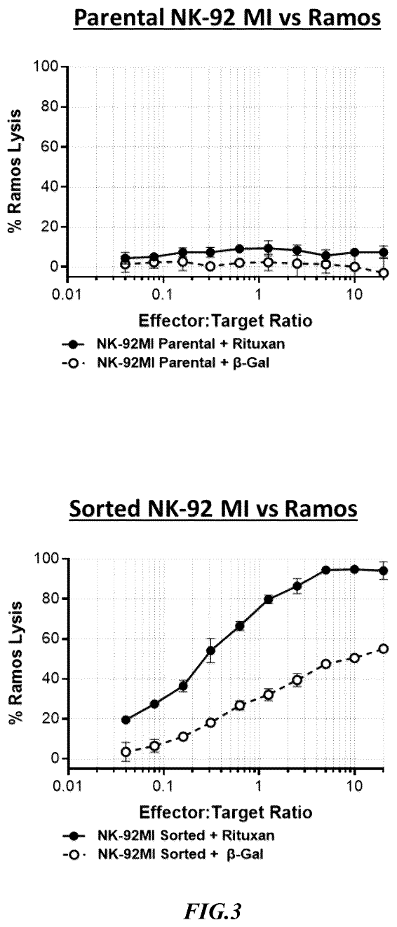

To examine if the sorted cells exert cytotoxicity against different cancer cell lines, parental or sorted NK-92MI were co-cultured with target cells like Ramos and HCC827 cells at different effector to target ratios in calcein AM based assay. As can be seen from FIG. 3, sorted but not parental cells showed enhanced direct cytotoxicity against Ramos cells (non-specific beta-gal treatment is comparable to direct cytotoxicity; 7% Ramos lysis with parental cell vs 55% Ramos lysis with sorted cells). FIG. 3 shows exemplary results for ADCC of sorted NK-92MI cells against Ramos target cells. More specifically, ADCC of parental (unsorted) and sorted NK-92MI cells against Ramos target cells in the presence of Rituxan was determined in a calcein release assay. Samples were tested in triplicate. Percentage (%) calcein release as an indicator of Target cell lysis is shown for different effector-to-target ratios.

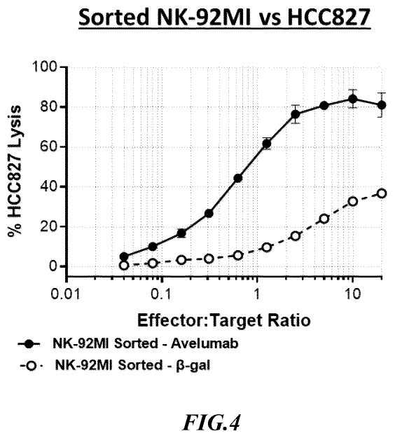

ADCC of parental and sorted NK-92MI cells was also tested against two tumor cell lines including HCC827 and Ramos cells in the presence of Avelumab and Rituximab respectively. As expected, parental cell lacking CD16 failed to induce ADCC mediated lysis of Ramos cells. In contrast, the sorted cells induced potent ADCC of Ramos cells (3% ADCC against Ramos by parental cell vs 94% ADCC against Ramos by sorted cells). Similar potent ADCC activity was observed when sorted cells were mixed with HCC827 cells in the presence of Avelumab as can be seen from the results in FIG. 4 depicting exemplary results for ADCC of sorted NK-92MI cells against HCC827 target cells. Here, ADCC of sorted NK-92MI cells against HCC827 target cells in the presence of Avelumab was determined in a calcein release assay. Samples were tested in triplicate. Percentage (%) calcein release as an indicator of Target cell lysis is shown for different effector-to-target ratios.

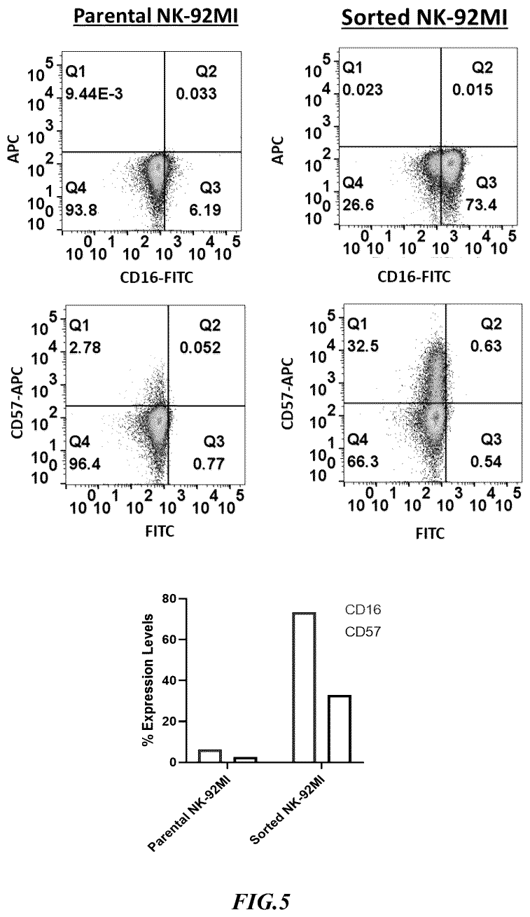

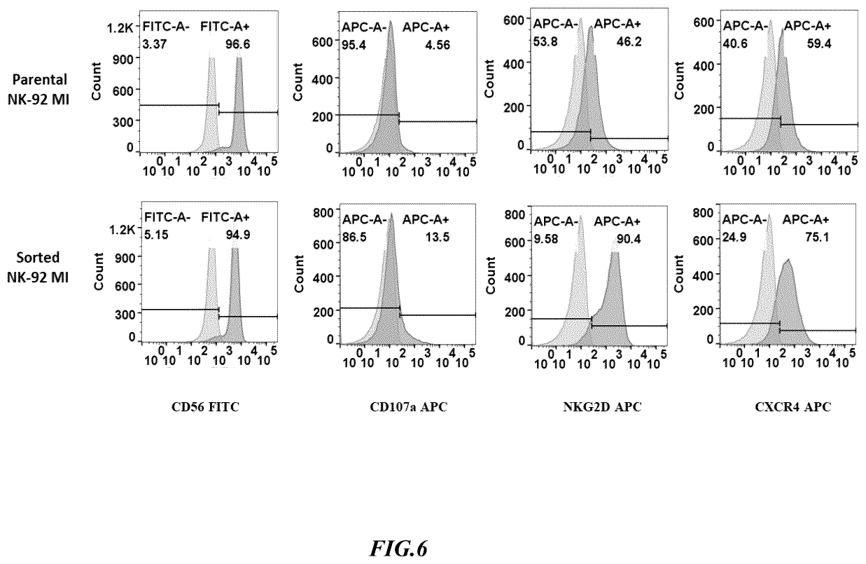

The expression stability was also evaluated after freeze thaw cycle. Sorted cells were frozen in ImmunityBio propriety media in the LN2 freezer. Post thaw and expansion, the sorted cells maintained the high expression of CD57, CD16 and NKG2D, and exemplary results are shown in FIG. 5 and FIG. 6. FIG. 5 depicts exemplary results establishing that NK-92mi sorted cells maintained high expression of CD57 and CD16 after freeze-thaw. Here, the surface marker expression on parental (unsorted) and sorted NK-92 MI cells was analyzed by flow cytometry-based staining and the results are presented below as dot blots. The bar graph compares the percent expression of respective markers on parental and sorted cells. FIG. 6 depicts exemplary results of surface phenotyping of parental and sorted NK-92MI cells after freeze-thaw. Here, the surface marker expression was analyzed by flow cytometry-based staining and the results are presented below as histogram. The light grey and dark grey indicate the marker staining on unstained and stained cells, respectively.

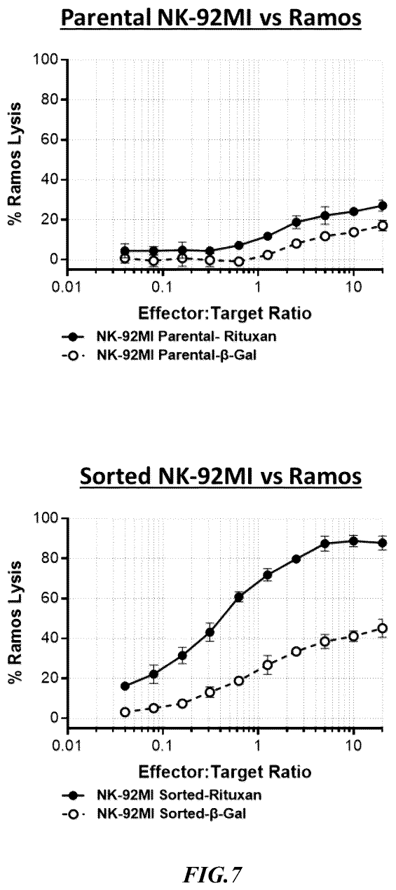

FIG. 7 shows exemplary results for ADCC of sorted NK-92MI cells after freeze-thaw. Here, ADCC of thawed NK-92MI cells against Ramos target cells in the presence of Rituxan was determined in a calcein release assay. Samples were tested in triplicate. Percentage (%) calcein release as an indicator of Target cell lysis is shown for different effector-to-target ratios. As can be seen, the freeze thaw cycle had no impact on CD16 mediated ADCC activity against CD20 expressing Ramos cells.

Based on the above, it should be recognized that the sorted cell line unexpectedly provided CD16+CD57+NK-92MI cell that natively expressed CD16 and CD57 and that exhibited IL-2 independent growth with significantly increased NKG2D in long term culture as well as after freeze-thaw.

For the experiments above, the inventors sorted CD57+CD16+NK cells from a parental NK-92MI, an IL-2 independent cell line. Approximately 60 million viable cells with greater than 90% viability were mixed with fluorescence labeled anti-CD16 and anti-CD57 antibodies for cell sorting using BD FACS Melody cell sorter. CD57+CD16+sorted NK cells were cultured up to 126 days in ImmunityBio specific media. On different days, flow cytometry analysis was performed using Miltenyi MACSQuant Analyzer 10, where cells were stained with fluorescent dye-conjugated antibody at room temperature for 10-30 min. The stained cells were washed and loaded up for flow cytometry analysis. Functional activity of the sorted cells was evaluated using a Calcein based method where sorted cells were mixed with calcein loaded tumor cells like Ramos cells in presence/absence of Rituxan antibody. NK cells induces the lysis of calcein-loaded target cells, leading to intracellular fluorescent calcein released into the assay medium. The level of fluorescence from the released calcein is directly proportional to the number of cells lysed and therefore a measure of cytotoxicity of the NK effector cells.

In still further experiments, the inventors conducted a gene expression analysis using mRNAseq in which the gene expression of genes was monitored across the cell sorting process. Notably, and among other findings, analysis of the mRNA seq on the sorted NK-92 MI CD16 cells revealed that there were about 50 genes (coding and non-coding) that showed significant sequential upregulation (over 4-fold) across the 2 steps sorting, and exemplary results are depicted in Table 1. As was expected, CD16a was one of the upregulated genes. Most of the remaining upregulated genes had functions in signal transduction, metabolism, and gene expression. Unexpectedly, Wnt6 and TRAIL DR1 were both highly enriched, with the role of Wnt6 being unclear, whereas the role of TRAIL DR1 would suggest an additional potential (indirect) mechanism of enhanced cytotoxicity.

TABLE 1
Significantly Upregulated Genes (p > 0.05)

| | Log2 Fold Change | Log2 Fold Change |
|---|---|---|
| Gene Name | After 1^{st} Sort | After 2^{nd} Sort |
| AC144831.1 | 2.99 | 4.57 |
| ADAMTS16 | 7.05 | 8.34 |
| BTG2 | 1.14 | 2.37 |
| CACNA2D1 | 2.18 | 3.69 |
| CAMK1D | 2.20 | 3.21 |
| CASP6 | 1.98 | 3.31 |
| CGNL1 | 4.43 | 6.09 |
| DAB2IP | 2.15 | 3.97 |
| DUSP16 | 1.86 | 3.02 |
| FAM96AP2 | 8.01 | 9.23 |
| FCGR3A | 1.65 | 4.05 |
| FOXP2 | 2.84 | 4.81 |
| FRG2B | 4.15 | 5.94 |
| GALK1 | 3.17 | 4.54 |
| GPD2 | 1.34 | 2.79 |
| GPR171 | 1.80 | 3.47 |
| GPRIN3 | 1.27 | 2.34 |
| IGF1 | 2.60 | 3.75 |
| KCNH8 | 3.96 | 5.41 |
| KIAA1147 | 1.40 | 2.85 |
| LINC01694 | 3.23 | 5.31 |
| LYSMD4 | 3.56 | 4.62 |
| MAN1C1 | 3.36 | 5.56 |
| MAP9 | 4.46 | 6.30 |
| MSC-AS1 | 2.07 | 3.63 |
| MYO18B | 4.14 | 5.85 |
| NA | 6.57 | 7.82 |
| NR5A2 | 6.34 | 7.75 |
| PCBP4 | 1.91 | 3.40 |
| PFKFB2 | 4.24 | 5.18 |
| PMCH | 4.66 | 6.15 |
| PRUNE2 | 2.38 | 3.38 |
| PURPL | 4.45 | 5.44 |
| RAPGEF5 | 3.38 | 4.38 |
| SGK1 | 2.22 | 3.45 |
| SIAH1P1 | 3.32 | 4.60 |
| SKI | 1.28 | 2.45 |
| SLC4A4 | 3.51 | 5.05 |
| SLC8B1 | 2.90 | 4.23 |
| SSX6 | 7.76 | 9.31 |
| ST8SIA5 | 7.37 | 9.33 |
| TCAF1 | 2.92 | 4.28 |
| TCIRG1 | 2.74 | 4.22 |
| TENM3 | 2.55 | 3.99 |
| TIAM1 | 1.78 | 3.02 |
| TLE2 | 3.59 | 4.85 |
| TNFRSF10A | 5.10 | 6.54 |
| USP43 | 7.46 | 9.36 |
| WNT6 | 5.39 | 6.45 |
| YPEL2 | 2.72 | 3.98 |

In addition, the inventors also identified 27 further genes (coding and non-coding) that showed significant sequential downregulation (over 4-fold) across the 2 steps sorting with most of these genes being generally involved in cell metabolism, and exemplary results are depicted in Table 2. Unexpectedly, PDGFD (platelet derived growth factor D) was among the significantly downregulated genes, which is a known “survival factor” in NK cells. Such finding was particularly notable as PDGFD is known as a potent stimulator of cell growth and motility that activates NK cells in an autocrine manner and contributes to interleukin-15-mediated NK cell survival. Such overexpression may contribute to the ability to expand the sorted cells in the absence of exogenous IL-2 and/or IL-15.

TABLE 2
Significantly Downregulated Genes (p > 0.05)

| | Log2 Fold Change | Log2 Fold Change |
|---|---|---|
| Gene Name | After 1^{st} Sort | After 2^{nd} Sort |
| AC124248.1 | −5.75 | −8.31 |
| AC244258.1 | −6.89 | −9.46 |
| AL031118.1 | −2.73 | −4.27 |
| AL773545.3 | −1.30 | −2.64 |
| ARHGEF11 | −1.75 | −3.19 |
| CYP11A1 | −3.35 | −7.06 |
| FAM30A | −1.94 | −3.02 |
| FER | −1.23 | −3.14 |
| GPT2 | −1.46 | −2.71 |
| HAGLROS | −2.87 | −4.05 |
| HIST3H2A | −1.36 | −2.96 |
| HIVEP3 | −1.63 | −2.75 |
| HTR2C | −5.60 | −8.17 |
| IGHM | −1.87 | −3.25 |
| MSR1 | −3.61 | −5.74 |
| OAS3 | −1.32 | −3.65 |
| PDGFD | −1.91 | −3.31 |
| PI16 | −2.01 | −3.11 |
| PTPRK | −2.53 | −4.28 |
| RNASE6 | −2.94 | −5.23 |
| SLC13A3 | −1.91 | −3.74 |
| SLC35F3 | −2.05 | −5.23 |
| SLC43A1 | −1.54 | −3.25 |
| SMIM24 | −1.94 | −3.91 |
| SNX20 | −1.62 | −2.71 |
| SPNS3 | −1.61 | −3.03 |
| STK32B | −1.89 | −3.54 |

The inventors further identified an additional 406 genes (coding and non-coding) that showed trends in upregulation during sorting. However, the data in upregulation were much more complex, with some genes showing upregulation after the 1st sorting step but not that much more after 2nd sorting, while other genes showed the opposite, with mild enrichment after 1st sorting but strong upregulation after 2nd sorting. Finally, further genes showed only mild enrichment after each sorting steps. Exemplary data are shown in Table 3. Notably, some of the upregulated genes included CD86, CD74, CD7, CCR5, CCR10, TIM3, KLRG1, SYK, IL21R, CXCR6, CD25, 4-1BB, GZMH, CD30, LAIR1, and NKG2D, which are believed to be associated with increased cytotoxic activity or chemotaxis. On the other hand, and somewhat unexpectedly, TIM3 and LAIR1 were upregulated, which are typically associated with decreased NK cells.

TABLE 3
Upregulated Genes

| | Log2 Fold Change | Log2 Fold Change |
|---|---|---|
| Gene Name | After 1^{st} Sort | After 2^{nd} Sort |
| ABCB1 | 0.97 | 1.86 |
| ABHD8 | 2.61 | 3.53 |
| AC002128.1 | 2.19 | 1.80 |
| AC002558.1 | 1.03 | 1.08 |
| AC003092.1 | 3.17 | 2.71 |
| AC004687.1 | 1.22 | 0.96 |
| AC005083.1 | 1.59 | 2.77 |
| AC005224.4 | 0.57 | 1.79 |
| AC005329.3 | 1.76 | 1.39 |
| AC007191.1 | 1.64 | 3.23 |
| AC007823.1 | 1.93 | 1.16 |
| AC008895.1 | 2.62 | 3.95 |
| AC009065.2 | 2.51 | 2.46 |
| AC009065.5 | 1.93 | 2.29 |
| AC009509.1 | 3.68 | 5.47 |
| AC010181.1 | 3.97 | 3.89 |
| AC010266.2 | 7.12 | 8.33 |
| AC010333.1 | 6.76 | 7.83 |
| AC016597.1 | 1.60 | 1.57 |
| AC016730.1 | 6.69 | 6.41 |
| AC017002.3 | 2.54 | 2.60 |
| AC017074.1 | 2.17 | 1.89 |
| AC022706.1 | 3.30 | 4.86 |
| AC024560.2 | 1.51 | 1.71 |
| AC046143.1 | 1.22 | 1.58 |
| AC073062.1 | 7.87 | 7.40 |
| AC078852.2 | 2.73 | 2.61 |
| AC087477.1 | 5.01 | 5.09 |
| AC087763.1 | 1.55 | 1.63 |
| AC090948.3 | 1.80 | 2.07 |
| AC092167.1 | 2.63 | 2.56 |
| AC092669.1 | 6.92 | 7.62 |
| AC093525.4 | 1.93 | 1.80 |
| AC097467.3 | 7.07 | 8.75 |
| AC098483.1 | 2.41 | 2.49 |
| AC100861.1 | 1.73 | 3.40 |
| AC103808.3 | 6.76 | 7.92 |
| AC106782.5 | 2.59 | 3.30 |
| AC109466.1 | 4.45 | 5.23 |
| AC111188.1 | 6.45 | 7.67 |
| AC114808.1 | 7.86 | 9.60 |
| AC130371.2 | 2.07 | 3.83 |
| AC135782.1 | 4.93 | 6.09 |
| AC138761.1 | 2.90 | 4.36 |
| AC142384.1 | 4.90 | 5.01 |
| AC145098.1 | 1.58 | 1.08 |
| AC233300.1 | 2.32 | 2.71 |
| ACP5 | 1.21 | 1.32 |
| ADAMTS1 | 3.89 | 4.79 |
| ADARB2 | 6.83 | 8.84 |
| ADGRG5 | 1.05 | 2.20 |
| ADRB2 | 3.78 | 3.26 |
| AFDN | 3.44 | 3.96 |
| AFF1 | 1.0 | 2.29 |
| AGRN | 3.43 | 4.81 |
| AKAP5 | 1.17 | 3.49 |
| AL031775.2 | 1.85 | 1.94 |
| AL035446.1 | 5.19 | 5.33 |
| AL121906.1 | 1.73 | 2.35 |
| AL121956.1 | 1.93 | 3.29 |
| AL133406.3 | 1.34 | 1.30 |
| AL138737.1 | 4.88 | 5.19 |
| AL161722.1 | 2.17 | 2.19 |
| AL359183.1 | 1.17 | 2.01 |
| AL390719.1 | 2.01 | 2.55 |
| AL645608.9 | 2.76 | 3.37 |
| ANK3 | 3.45 | 4.72 |
| ANKRD20A11P | 1.51 | 2.32 |
| ANKRD20A9P | 2.83 | 2.87 |
| ANKRD55 | 0.08 | 1.78 |
| ANO1 | 3.45 | 7.96 |
| ANO10 | 1.55 | 2.44 |
| ANXA5 | 1.16 | 1.48 |
| ANXA9 | 3.08 | 5.10 |
| AOC1 | 4.10 | 6.54 |
| AP001107.6 | 1.01 | 1.50 |
| AP001458.1 | 1.66 | 1.22 |
| AP001783.1 | 5.89 | 6.56 |
| AP002807.1 | 3.68 | 5.14 |
| AREG | 2.6 | 2.53 |
| ARHGAP12 | 1.76 | 2.91 |
| ARHGAP31 | 4.13 | 5.66 |
| ARHGEF26 | 5.17 | 7.07 |
| ARHGEF26-AS1 | 2.30 | 3.56 |
| ARID5A | 4.33 | 5.19 |
| ATN1 | 0.71 | 1.50 |
| AXIN1 | 1.17 | 1.75 |
| BATF3 | 1.28 | 0.94 |
| BMI1 | 1.48 | 2.15 |
| BMPR1B | 2.39 | 1.39 |
| BTBD3 | 3.27 | 3.43 |
| C11orf44 | 3.2 | 3.43 |
| CIQA | 7.62 | 8.22 |
| C3AR1 | 1.16 | 2.27 |
| CABYR | 1.63 | 1.50 |
| CACNA2D3 | 4.81 | 4.21 |
| CACNB4 | 3.57 | 3.18 |
| CASC18 | 1.87 | 2.61 |
| CATSPER1 | 1.81 | 2.86 |
| CBFA2T3 | 1.37 | 3.61 |
| CCR10 | 2.41 | 3.16 |
| CCR5 | 1.14 | 2.02 |
| CD151 | 1.22 | 1.60 |
| CD40LG | 4.68 | 4.66 |
| CD6 | 0.94 | 3.82 |
| CD7 | 2.78 | 3.85 |
| CD74 | 5.17 | 6.77 |
| CD82 | 1.36 | 1.75 |
| CD86 | 2.54 | 4.96 |
| CDH19 | 4.50 | 4.36 |
| CDH9 | 4.39 | 6.54 |
| CDK14 | 4.21 | 4.94 |
| CFAP61 | 3.57 | 4.01 |
| CHCHD3P3 | 1.25 | 1.12 |
| CHD3 | 0.69 | 1.73 |
| CHST10 | 0.10 | 4.21 |
| CLC | 5.54 | 4.50 |
| CLSTN2 | 5.57 | 5.73 |
| CLVS2 | 4.1 | 4.28 |
| CMKLR1 | 0.55 | 3.01 |
| CMTM5 | 4.32 | 5.19 |
| COL9A2 | 4.82 | 6.30 |
| CORO2A | 1.53 | 3.84 |
| CPEB4 | 0.49 | 2.00 |
| CPNE3 | 1.23 | 0.78 |
| CRYZP1 | 2.45 | 2.32 |
| CSF | 1.11 | 1.65 |
| CSGALNACT1 | 1.47 | 2.42 |
| CST4 | 8.09 | 7.68 |
| CXCR6 | 1.85 | 2.24 |
| CYP4F29P | 2.19 | 1.57 |
| CYP4F34P | 3.53 | 3.84 |
| D2HGDH | 1.17 | 1.27 |
| DAPP1 | 2.46 | 2.66 |
| DCBLD2 | 0.48 | 1.71 |
| DMD | 4.54 | 4.08 |
| DNTT | 7.01 | 9.39 |
| DTHD1 | 3.88 | 6.44 |
| DUSP23 | 1.74 | 2.48 |
| DUSP3 | 1.39 | 3.11 |
| DUXB | 3.27 | 5.70 |
| EBF1 | 4.97 | 5.61 |
| EEPD1 | 2.90 | 1.97 |
| EGR2 | 3.03 | 3.12 |
| EIF5 | 1.67 | 1.02 |
| ELAVL2 | 0.41 | 2.86 |
| ENDOD1 | 1.30 | 0.56 |
| ENGASE | 1.94 | 3.56 |
| ENTPD1 | 0.15 | 1.19 |
| EPB41L3 | 2.00 | 2.72 |
| EPHB6 | 2.63 | 2.12 |
| EPS15P1 | 1.25 | 1.41 |
| ERC1 | 0.74 | 2.11 |
| EYA4 | 0.44 | 2.30 |
| F13A1 | 5.03 | 7.34 |
| F2RL3 | 2.10 | 2.55 |
| FAM166B | 3.21 | 3.87 |
| FAM96AP2 | 8.01 | 9.23 |
| FAS | 0.61 | 1.47 |
| FBXL7 | 3.02 | 2.43 |
| FBXO32 | 1.68 | 3.05 |
| FCHSD1 | 1.54 | 2.16 |
| FCRL3 | 2.24 | 2.86 |
| FGL2 | 0.62 | 2.12 |
| FLNA | 1.06 | 1.84 |
| FLRT3 | 1.76 | 2.25 |
| FRG2 | 1.93 | 1.97 |
| FRG2EP | 2.19 | 3.75 |
| FRMPD3 | 3.03 | 6.29 |
| GABRB3 | 1.64 | 0.93 |
| GADD45B | 1.25 | 1.04 |
| GATA6 | 6.75 | 6.26 |
| GBP2 | 1.15 | 1.41 |
| GCSAM | 4.75 | 5.07 |
| GDF11 | 1.01 | 2.15 |
| GGT1 | 1.13 | 2.09 |
| GOLGA7B | 2.04 | 2.22 |
| GORASP1 | 1.13 | 2.29 |
| GRIN2A | 7.92 | 9.76 |
| GUCY1A1 | 2.51 | 3.42 |
| GZMB | 1.30 | 0.58 |
| GZMH | 2.65 | 2.68 |
| HAPLN3 | 2.95 | 3.61 |
| HAVCR1 | 2.19 | 3.46 |
| HAVCR2 | 2.35 | 2.87 |
| HCN3 | 1.40 | 2.29 |
| HGF | 2.69 | 2.25 |
| HIGD1B | 1.34 | 1.80 |
| HLA-DPA1 | 4.43 | 4.40 |
| HLA-DRA | 3.85 | 3.67 |
| HNF4G | 2.12 | 1.80 |
| HNRNPA1P8 | 2.89 | 4.42 |
| HOXC6 | 2.46 | 2.06 |
| HSD17B7P2 | 2.67 | 3.94 |
| HSPA8 | 1.07 | 0.59 |
| HYLS1 | 2.19 | 3.57 |
| IFFO2 | 0.37 | 1.42 |
| IL10 | 1.24 | 0.30 |
| IL21R | 2.10 | 2.60 |
| IL2RA | 1.35 | 1.64 |
| ILDR1 | 3.12 | 4.71 |
| IMPG1 | 2.47 | 3.57 |
| INPP5F | 0.62 | 1.87 |
| INSC | 1.00 | 0.32 |
| IRF4 | 1.38 | 1.64 |
| IRF5 | 0.67 | 5.46 |
| ISLR | 2.19 | 2.94 |
| JAK3 | 0.81 | 1.58 |
| JUNB | 1.59 | 1.22 |
| KCND2 | 3.47 | 3.23 |
| KCP | 0.84 | 3.05 |
| KCTD11 | 0.94 | 2.26 |
| KIAA0930 | 1.20 | 2.46 |
| KIAA1217 | 3.53 | 4.21 |
| KIAA1841 | 0.90 | 2.18 |
| KIF16B | 7.13 | 8.13 |
| KIF1B | 2.30 | 2.89 |
| KLHL1 | 2.91 | 2.48 |
| KLK1 | 4.78 | 5.95 |
| KLRAIP | 2.17 | 3.44 |
| KLRG1 | 1.52 | 2.87 |
| KLRK1 | 0.87 | 2.01 |
| LAIR1 | 0.49 | 2.24 |
| LAPTM5 | 1.03 | 2.01 |
| LBH | 0.63 | 1.86 |
| LFNG | 1.50 | 2.15 |
| LHFPL2 | 2.46 | 2.47 |
| LIN7A | 4.20 | 3.80 |
| LINC00324 | 1.03 | 2.95 |
| LINC00408 | 4.83 | 4.97 |
| LINC00431 | 2.90 | 3.52 |
| LINC00877 | 4.38 | 4.98 |
| LINC00892 | 4.19 | 4.86 |
| LINC00923 | 3.93 | 3.34 |
| LINC01010 | 3.37 | 4.17 |
| LINC01132 | 2.40 | 2.83 |
| LINC01140 | 2.47 | 2.27 |
| LINC01821 | 2.23 | 2.38 |
| LINC01943 | 4.07 | 3.18 |
| LINC02147 | 7.69 | 8.19 |
| LINC02245 | 5.88 | 6.43 |
| LINC02254 | 4.17 | 4.75 |
| LINC02484 | 2.92 | 3.05 |
| LMNA | 1.02 | 1.50 |
| LMO3 | 3.10 | 6.56 |
| LPXN | 1.03 | 2.14 |
| LRP11 | 2.31 | 2.86 |
| LRRN3 | 2.52 | 3.29 |
| LSS | 0.92 | 1.46 |
| MACC1 | 3.47 | 4.63 |
| MACROD2 | 2.48 | 2.92 |
| MAFF | 1.91 | 2.14 |
| MAGEA6 | 2.73 | 4.19 |
| MALAT1 | 1.27 | 1.57 |
| MAN2A2 | 0.91 | 2.02 |
| MAPK8 | 1.27 | 1.56 |
| MED13L | 0.63 | 1.50 |
| METRNL | 1.72 | 2.41 |
| METTL15P3 | 5.95 | 5.33 |
| METTL7A | 0.75 | 1.97 |
| MFHAS1 | 1.42 | 3.35 |
| MIAT | 3.14 | 4.41 |
| MIR4326 | 1.37 | 0.99 |
| MIR93 | 2.51 | 2.68 |
| MLC1 | 1.38 | 2.32 |
| MS4A1 | 5.22 | 5.70 |
| MSC | 3.41 | 4.24 |
| MT-CYB | 1.06 | 0.66 |
| MUC1 | 1.73 | 3.74 |
| MYH7 | 5.98 | 5.97 |
| MYO10 | 3.60 | 4.50 |
| MYPN | 3.63 | 3.94 |
| NA | 4.43 | 4.05 |
| NAMPT | 1.17 | 0.61 |
| NCOR1P2 | 8.15 | 9.73 |
| NCS1 | 0.98 | 2.61 |
| NDRG1 | 0.51 | 1.92 |
| NEAT1 | 1.23 | 1.33 |
| NEU1 | 1.24 | 1.07 |
| NOTCH1 | 1.08 | 1.97 |
| NOTCH2 | 1.21 | 1.50 |
| NOX3 | 7.78 | 9.03 |
| NR4A2 | 0.36 | 2.38 |
| NTRK2 | 4.69 | 6.08 |
| NUAK1 | 2.16 | 2.95 |
| OLFM1 | 1.73 | 0.94 |
| OSBPL10 | 0.37 | 2.96 |
| OSM | 2.39 | 1.67 |
| PACS1 | 0.33 | 2.01 |
| PAX5 | 1.69 | 0.71 |
| PCBP3 | 1.59 | 3.85 |
| PDE2A | 2.43 | 1.86 |
| PDE4DIP | 1.43 | 0.52 |
| PDE7B | 1.80 | 2.54 |
| PEX5L | 2.66 | 3.29 |
| PGAMIP8 | 1.01 | 0.97 |
| PHF20L1 | 1.12 | 2.16 |
| PHLDA1 | 2.97 | 3.04 |
| PIK3R6 | 5.65 | 4.77 |
| PIM2 | 1.40 | 1.00 |
| PKD1L1 | 2.32 | 2.26 |
| PKIB | 3.47 | 3.70 |
| PKP4-AS1 | 3.32 | 2.39 |
| PLCB1 | 1.04 | 2.66 |
| PLCB4 | 1.11 | 2.57 |
| PLEKHB1 | 2.21 | 3.43 |
| PLIN3 | 0.69 | 1.99 |
| PLS3 | 4.07 | 4.86 |
| PLXNA3 | 1.56 | 2.14 |
| PLXNA4 | 1.36 | 0.52 |
| POF1B | 5.31 | 6.62 |
| POLR2J4 | 1.30 | 2.37 |
| PPARA | 0.57 | 2.12 |
| PPM1H | 3.13 | 3.01 |
| PRDM9 | 7.63 | 6.85 |
| PRKCD | 0.80 | 1.97 |
| PROCR | 1.05 | 0.92 |
| PROSER2 | 2.22 | 6.28 |
| PSTPIP2 | 2.25 | 3.16 |
| PTN | 6.50 | 6.05 |
| PTPDC1 | 2.03 | 2.24 |
| PTPN12 | 1.04 | 1.75 |
| PTPRG | 4.22 | 5.56 |
| RAB20 | 3.67 | 3.13 |
| RAB27B | 0.62 | 2.17 |
| RAPIGDS1 | 0.98 | 1.74 |
| RCAN2 | 4.02 | 3.92 |
| REC8 | 1.34 | 1.86 |
| RF00017 | 3.10 | 2.61 |
| RF00019 | 2.73 | 2.61 |
| RHOB | 1.90 | 1.29 |
| RHOTIP3 | 3.44 | 3.90 |
| RIMS1 | 5.71 | 4.97 |
| RNU5A-1 | 2.51 | 2.61 |
| S100A10 | 1.11 | 1.54 |
| SATB1 | 1.12 | 1.82 |
| SBK1 | 0.57 | 1.91 |
| SCART1 | 2.47 | 1.50 |
| SCD5 | 1.16 | 2.41 |
| SCN3A | 1.32 | 1.35 |
| SDC4 | 3.71 | 4.63 |
| SEMA3A | 4.14 | 4.72 |
| SEMA4A | 1.72 | 1.21 |
| SEMA5A | 2.25 | 1.39 |
| SHROOM2 | 4.83 | 8.42 |
| SLAMF1 | 3.58 | 3.79 |
| SLC12A8 | 2.35 | 3.02 |
| SLC1A3 | 3.51 | 4.16 |
| SLC52A2 | 1.40 | 1.72 |
| SMARCAD1 | 0.80 | 1.69 |
| SMOX | 1.46 | 4.70 |
| SNHG12 | 1.28 | 0.69 |
| SNX19P2 | 2.47 | 2.20 |
| SORBS2 | 2.93 | 3.61 |
| SOX2-OT | 2.69 | 1.98 |
| SOX5 | 4.83 | 4.23 |
| SPACA3 | 1.29 | 1.52 |
| SPINK5 | 3.86 | 4.26 |
| SPOCK1 | 0.65 | 2.29 |
| SSX1 | 8.55 | 10.09 |
| SSX6 | 7.76 | 9.31 |
| ST8SIA5 | 7.37 | 9.33 |
| ST8SIA6 | 3.65 | 4.96 |
| STBD1 | 1.50 | 1.74 |
| STRIP2 | 1.61 | 1.12 |
| SYK | 1.27 | 1.82 |
| TBXAS1 | 6.91 | 7.95 |
| TCAF1P1 | 2.96 | 4.26 |
| TCAF2 | 1.06 | 1.47 |
| TESK2 | 1.04 | 2.42 |
| TEX11 | 1.92 | 2.38 |
| TIGD7 | 1.45 | 1.39 |
| TIMD4 | 3.44 | 3.65 |
| TIMP1 | 1.24 | 1.93 |
| TMC1 | 4.10 | 5.46 |
| TMEM255B | 2.93 | 3.02 |
| TMEM71 | 3.73 | 4.32 |
| TNFRSF11A | 6.08 | 5.95 |
| TNFRSF14-AS1 | 0.94 | 1.49 |
| TNFRSF19 | 1.69 | 1.80 |
| TNFRSF8 | 4.83 | 4.68 |
| TNFRSF9 | 5.77 | 5.96 |
| TNIP3 | 1.44 | 1.59 |
| TNS1 | 0.95 | 2.02 |
| TRBV12-5 | 1.66 | 2.39 |
| TRDN | 1.12 | 1.80 |
| TRGV6 | 2.12 | 2.49 |
| TRIB1 | 2.14 | 2.18 |
| TRPS1 | 1.38 | 2.52 |
| TSPAN33 | 1.54 | 1.83 |
| TTYH3 | 0.12 | 2.03 |
| USH2A | 2.01 | 2.12 |
| USP43 | 7.46 | 9.36 |
| VAT1 | 0.58 | 2.40 |
| VDR | 5.76 | 5.30 |
| WNK1 | 0.42 | 1.34 |
| YBX3 | 3.33 | 4.13 |
| Z93241.1 | 3.17 | 3.32 |
| ZC3H12C | 1.97 | 2.68 |
| ZNF252P | 0.97 | 2.08 |
| ZNF284 | 2.51 | 4.05 |
| ZNF350 | 3.93 | 3.80 |
| ZNF623 | 0.65 | 2.00 |
| ZNF680 | 4.18 | 4.81 |
| ZNF683 | 3.41 | 2.92 |
| ZYX | 1.12 | 1.22 |

Finally, the inventors also identified about 350 genes (coding and non-coding) that showed trends in downregulation during sorting as can be seen from the data in Table 4. As above, the picture is complex. Most notably, downregulated genes that are typically associated with cytotoxicity in NK cells were CD11c, CD11d, IL25, NKp44, and CD28. Most unexpectedly, however, the expression of the IL-2 gene was substantially downregulated with serial sequencing (by almost 10-fold).

TABLE 4
Downregulated Genes

| | Log2 Fold Change | Log2 Fold Change |
|---|---|---|
| Gene Name | After 1^{st} Sort | After 2^{nd} Sort |
| ABCA4 | −3.72 | −4.84 |
| ABHD15-AS1 | −0.16 | −2.29 |
| ABLIM1 | −1.09 | −2.04 |
| AC002470.1 | −2.16 | −1.70 |
| AC002480.1 | −1.15 | −1.64 |
| AC002480.2 | −1.02 | 1.88 |
| AC004585.1 | −1.10 | −0.89 |
| AC010531.1 | −0.25 | −1.48 |
| AC012447.1 | −0.62 | −2.45 |
| AC016583.1 | −1.11 | −3.48 |
| AC016687.2 | −0.72 | −1.94 |
| AC016876.1 | −1.17 | −2.06 |
| AC018552.2 | −4.19 | −4.31 |
| AC021851.1 | 3.47 | 3.31 |
| AC078850.2 | −1.32 | −3.22 |
| AC084759.2 | −2.28 | −7.21 |
| AC087857.1 | −1.44 | −7.60 |
| AC090204.1 | −0.71 | −2.98 |
| AC093520.1 | −1.92 | −3.08 |
| AC096631.2 | −4.24 | −4.95 |
| AC098936.1 | −3.40 | −4.37 |
| AC104958.1 | −0.69 | −2.99 |
| AC108134.1 | −0.82 | −2.69 |
| AC116359.1 | −2.13 | −5.84 |
| AC119396.1 | −1.11 | −2.69 |
| AC128689.1 | −2.07 | 7.81 |
| AC135068.6 | −0.42 | −1.20 |
| AC243965.1 | 0.75 | −1.88 |
| AC245100.3 | −3.60 | −5.72 |
| AC245297.1 | −0.38 | −0.73 |
| ACKR3 | −2.12 | −2.80 |
| ACOXL | −0.87 | −2.04 |
| ADAM28 | −0.78 | −3.11 |
| ADGRB2 | −0.66 | −1.33 |
| ADGRG3 | −1.43 | −3.39 |
| ADGRL2 | 0.07 | −0.23 |
| AFAPIL2 | −1.04 | 1.59 |
| AGTRAP | −1.15 | −2.87 |
| AIF1 | −0.79 | −3.24 |
| AL008721.1 | 0.01 | 1.29 |
| AL021807.1 | −0.41 | −3.25 |
| AL034376.1 | −0.93 | −3.45 |
| AL034376.2 | −0.38 | −2.75 |
| AL109761.1 | −1.17 | −1.30 |
| AL121904.1 | −2.99 | −8.37 |
| AL121983.1 | −1.75 | −1.88 |
| AL132640.1 | −0.94 | −1.94 |
| AL139125.1 | −0.40 | −2.07 |
| AL157813.1 | −0.92 | −2.06 |
| AL159166.1 | −1.52 | −3.80 |
| AL161729.2 | −0.35 | 1.29 |
| AL163051.1 | −0.68 | −2.53 |
| AL353803.1 | −1.50 | −5.27 |
| AL355512.1 | −1.76 | −3.55 |
| AL355994.2 | −1.44 | −1.78 |
| AL357037.1 | −2.16 | −2.29 |
| AL358473.1 | −1.34 | −3.17 |
| AL450468.2 | 0.37 | −1.20 |
| AL591846.1 | −0.30 | −1.37 |
| AL662890.1 | −1.27 | −1.88 |
| ALKBH3 | −0.54 | −2.53 |
| ALOXE3 | −1.16 | 1.96 |
| ANGPT4 | −0.84 | −2.92 |
| ANKEF1 | 0.03 | −2.03 |
| AP1S3 | −0.85 | −2.36 |
| APOBEC3H | −1.01 | −1.98 |
| ASB12 | −1.40 | −3.52 |
| ATF4 | −1.01 | −1.95 |
| ATP2C2 | −0.54 | −1.37 |
| ATP8A2 | −0.57 | −1.48 |
| BCAR3 | −1.77 | 2.83 |
| BCHE | −0.59 | −1.61 |
| BCL2L11 | −0.80 | −2.11 |
| BIVM | −0.64 | −2.28 |
| BLNK | −0.84 | 1.96 |
| BTBD6P1 | −0.26 | −0.77 |
| BX322234.1 | −1.22 | −2.08 |
| C10orf25 | −1.75 | −2.20 |
| C10orf91 | −0.09 | −2.14 |
| C15orf41 | −0.84 | −2.12 |
| Clorf198 | −1.01 | −2.52 |
| C4orf48 | −0.81 | −3.06 |
| C8B | −0.84 | −7.10 |
| CA6 | −2.92 | −3.84 |
| CAPN8 | −2.13 | −3.04 |
| CARD17 | −1.70 | −5.36 |
| CASP1P2 | −0.93 | −3.63 |
| CATSPERB | −0.77 | −1.78 |
| CCDC113 | −0.88 | −2.72 |
| CCDC171 | −1.14 | −3.01 |
| CCR3 | −0.36 | −2.68 |
| CCRL2 | −0.53 | −1.10 |
| CD101 | −2.81 | −3.55 |
| CD28 | −1.07 | −3.48 |
| CD80 | −0.77 | −0.98 |
| CDCP1 | −1.07 | −1.60 |
| CDHR1 | −1.27 | −4.41 |
| CDX1 | −3.42 | −4.55 |
| CENPV | −0.72 | 3.22 |
| CEP112 | −1.45 | −3.56 |
| CHD7 | −0.37 | −2.41 |
| CHRD | −0.51 | −3.34 |
| CKS1BP2 | −0.81 | −1.52 |
| CLIP2 | −1.97 | 1.99 |
| CPNE7 | −0.91 | −2.71 |
| CPS1 | 0.03 | −1.75 |
| CR382285.1 | −1.00 | −2.13 |
| CRTAP | −1.15 | −2.01 |
| CRYBA4 | −2.05 | −2.81 |
| CSAG1 | −2.00 | −2.86 |
| CYSLTR1 | −1.58 | −5.07 |
| DGKQ | −0.74 | −1.91 |
| DMAC1 | −0.47 | −2.13 |
| DNAI1 | −1.49 | −1.61 |
| DOK2 | −2.55 | −3.46 |
| DSC1 | −1.46 | −4.56 |
| DSC3 | −2.19 | −5.90 |
| DYNC1I1 | −2.74 | −2.99 |
| DYSF | −0.55 | −0.86 |
| EIF4EBP1 | −0.83 | 2.48 |
| EPSTI1 | −0.91 | −2.14 |
| ERLIN2 | −0.91 | −2.11 |
| FAM107B | −1.30 | −1.79 |
| FAM160A1 | −1.22 | −4.29 |
| FAM227A | −0.27 | −1.20 |
| FES | −0.27 | −2.16 |
| FGD2 | −0.43 | −1.97 |
| FMN2 | −0.82 | −1.74 |
| FMNL2 | −0.66 | −0.48 |
| FREM3 | −0.40 | −1.11 |
| FUT7 | −0.81 | −2.65 |
| GABBR2 | −0.70 | −1.48 |
| GABRR2 | −1.46 | −3.17 |
| GADD45A | −1.31 | −1.36 |
| GIMAP4 | −0.73 | −2.06 |
| GLDC | −0.59 | −2.40 |
| GNAO1 | −1.56 | −0.71 |
| GNAS-AS1 | −0.84 | −3.00 |
| HACD4 | −0.59 | −2.51 |
| HAGLR | −1.55 | −4.30 |
| HAPLN1 | −1.32 | −3.45 |
| HBEGF | −0.49 | −2.64 |
| HCG17 | −1.35 | −2.29 |
| HEPH | −0.66 | −3.58 |
| HICI | −1.56 | 0.84 |
| HIST1H1C | −0.04 | −2.82 |
| HIST1H2AC | −0.20 | −2.67 |
| HIST1H2AL | 0.00 | −2.35 |
| HIST1H2BJ | −0.47 | −2.61 |
| HIST1H2BO | −0.67 | −3.46 |
| HIST2H2BE | −0.56 | −2.61 |
| HJURP | −1.08 | −1.26 |
| HMGN5 | 1.42 | −1.12 |
| HOMER2P1 | −1.21 | −2.28 |
| HVCN1 | −1.26 | −1.73 |
| IFI27 | −4.28 | −8.06 |
| IFI44 | −0.91 | −3.13 |
| IFI44L | −1.55 | −5.18 |
| IFIT1 | −1.56 | −2.58 |
| IFIT2 | −1.25 | −2.77 |
| IFIT3 | −1.22 | −3.32 |
| IFNG-AS1 | −0.49 | −2.22 |
| IGHD | −3.15 | −3.78 |
| IGHG3 | −0.64 | −4.59 |
| IGHJ1 | −5.16 | −7.73 |
| IGHJ3 | −4.53 | −8.10 |
| IL1RN | −0.83 | −3.02 |
| IL2 | −0.92 | −3.31 |
| IL25 | −2.92 | −5.37 |
| IL26 | −0.35 | −1.70 |
| INAVA | −0.15 | −2.85 |
| INPP5A | −0.58 | −0.83 |
| IRF8 | −0.63 | −2.01 |
| ITGAD | −2.32 | −3.20 |
| ITGAX | −1.88 | −1.81 |
| KALRN | −2.44 | −3.37 |
| KDELC1 | −0.96 | −2.14 |
| KIR2DS4 | −1.28 | −1.46 |
| KIR3DL1 | −0.43 | −2.60 |
| KLHL13 | −2.04 | −3.02 |
| KLHL9 | −1.26 | −1.62 |
| KLRF2 | −0.32 | −0.25 |
| L3MBTL1 | −0.88 | −2.73 |
| LAMA3 | −1.60 | −3.22 |
| LDB2 | −0.61 | −1.40 |
| LEPR | −0.58 | −1.01 |
| LGALS3BP | −0.87 | −0.47 |
| LGALS9B | −1.21 | −2.78 |
| LHPP | −1.09 | −1.81 |
| LIMS2 | −2.10 | −3.51 |
| LIN28B | −1.12 | −2.94 |
| LINC00558 | −0.40 | −2.52 |
| LINC00861 | −1.17 | −2.97 |
| LINC01146 | −2.07 | −4.31 |
| LINC01206 | −0.16 | −0.36 |
| LINC01593 | −2.80 | −8.69 |
| LINC01594 | −3.07 | −5.52 |
| LINC01644 | −0.56 | −1.91 |
| LINC01819 | −2.97 | −5.90 |
| LINC02054 | −1.23 | −3.52 |
| LINC02237 | −1.22 | 4.23 |
| LINC02273 | −1.06 | −3.38 |
| LINC02362 | −0.70 | −2.88 |
| LINC02475 | −5.03 | −7.60 |
| LINGO2 | −0.91 | −3.41 |
| LONRF1 | −0.41 | −1.55 |
| LPAR3 | −0.79 | −3.07 |
| LRFN3 | −0.32 | −1.06 |
| LZTS1 | −0.78 | −1.32 |
| MAF | −0.65 | −2.61 |
| MAGEH1 | −1.24 | −1.13 |
| MATN4 | −2.88 | −9.97 |
| MEF2C | −0.46 | −1.50 |
| MEGF6 | −1.40 | −1.52 |
| MIR4537 | −1.22 | −1.91 |
| MIR4538 | −1.07 | −2.18 |
| MIR4539 | −1.53 | −2.22 |
| MLLT3 | −1.16 | −1.61 |
| MMP25 | −0.78 | −2.02 |
| MMP9 | −0.56 | −1.07 |
| MPP2 | −0.61 | −0.83 |
| MTAP | −0.73 | −3.18 |
| MTTP | 0.98 | −4.35 |
| MX1 | −1.03 | −2.90 |
| MYBPH | −1.28 | −3.28 |
| MYL5 | −0.70 | −2.28 |
| MYO16 | −0.95 | −2.25 |
| MYO7B | −1.50 | −2.41 |
| MYOC | −0.85 | −2.81 |
| MYT1 | 0.26 | −0.65 |
| NA | −0.56 | −1.70 |
| NBPF17P | −5.42 | −5.96 |
| NCF4 | −0.65 | −0.79 |
| NCKAP5 | −0.17 | −1.30 |
| NCR2 | −1.49 | −2.05 |
| NDST1 | −0.68 | −0.46 |
| NDUFAF2 | −0.34 | −1.80 |
| NEDD4L | −0.22 | −2.09 |
| NEK11 | −1.00 | −2.13 |
| NEXN | −0.89 | −3.94 |
| NHSL2 | −2.73 | −2.66 |
| NKX3-1 | −0.96 | −1.60 |
| NLRP11 | −1.00 | −2.71 |
| NPM3 | −0.33 | −1.73 |
| NRROS | −0.98 | −1.91 |
| NTM | −0.91 | −3.45 |
| OAS1 | −1.03 | −2.13 |
| OPN3 | −1.38 | −2.13 |
| OR2B6 | −0.54 | −3.49 |
| OSBPL1A | −0.91 | −1.90 |
| PALLD | −2.20 | −1.73 |
| PCDH10 | −0.75 | −2.68 |
| PCK2 | −0.91 | −1.79 |
| PCSK5 | −1.04 | −2.91 |
| PELO | −0.35 | −1.24 |
| PHACTRI | −0.20 | −0.77 |
| PIEZO2 | −0.87 | −2.31 |
| PKD1L3 | −0.08 | −0.52 |
| PLAG1 | −1.21 | −2.50 |
| PLIN2 | −0.99 | −3.45 |
| PLOD2 | −0.41 | −4.42 |
| PMAIP1 | −0.93 | −2.29 |
| PNPLA7 | −0.13 | −1.64 |
| POLR3G | −0.27 | −1.95 |
| PPARGC1A | −1.73 | −4.97 |
| PPP4R4 | −3.07 | −3.20 |
| PROM1 | −1.54 | −3.20 |
| PTAFR | −3.74 | −3.79 |
| PTGDR | −1.54 | −0.74 |
| PTK7 | −0.35 | 1.67 |
| PTPRM | −1.14 | −1.18 |
| RALGAPA2 | −0.67 | −0.78 |
| RASEF | −1.10 | −2.65 |
| RASGEF1B | −1.78 | −3.62 |
| RASGRP3 | −0.24 | −0.52 |
| RASSF4 | −0.94 | −0.45 |
| RBM41 | −0.41 | −2.04 |
| RBPJL | −2.77 | −4.58 |
| RDM1 | −1.33 | −1.87 |
| RF00019 | −0.40 | −1.11 |
| RGL4 | −0.83 | −2.08 |
| RIPOR2 | 1.80 | −2.81 |
| RPL17 | −0.28 | −1.96 |
| RPL24P2 | −0.17 | −1.30 |
| RPS6 | −0.37 | −1.83 |
| RYK | −0.94 | 2.33 |
| SCAMP1-AS1 | −1.35 | −1.62 |
| SCIMP | −0.81 | −1.52 |
| SCNN1B | −2.14 | −4.59 |
| SDK2 | −0.90 | −2.62 |
| SESN2 | −1.16 | −1.10 |
| SESTD1 | −1.20 | −1.98 |
| SETD7 | −1.11 | −3.03 |
| SIGLEC6 | −0.35 | −2.08 |
| SIRPG | −0.48 | −1.23 |
| SLC22A31 | −0.31 | −0.98 |
| SLC24A2 | −3.12 | −5.71 |
| SLC24A4 | −0.99 | −2.63 |
| SLC7A5 | −0.52 | −2.01 |
| SLCO4C1 | −0.14 | −0.51 |
| SNPH | −0.61 | −1.61 |
| SNTG1 | −0.41 | −1.48 |
| SNX7 | 0.01 | −2.29 |
| SORCS2 | −0.77 | −1.88 |
| SORD | −3.79 | −4.18 |
| SPOCK2 | −0.70 | −2.2 |
| SRSF10P1 | −0.84 | −3.87 |
| SSC5D | −0.50 | −1.21 |
| ST18 | −0.07 | 4.35 |
| STK32C | −1.26 | −2.73 |
| STRADB | −0.47 | −2.20 |
| SVEP1 | −2.62 | −4.86 |
| SYNPO | −0.15 | −0.52 |
| TC2N | −1.09 | −1.79 |
| TCAMIP | −0.79 | −3.67 |
| TDO2 | −1.34 | −5.59 |
| TESPA1 | −0.24 | −2.05 |
| TEX19 | −0.54 | −2.23 |
| THBS2 | −1.09 | −2.06 |
| THPO | −0.07 | −2.30 |
| TJP1 | −0.58 | −2.05 |
| TMEM170B | −1.05 | −2.13 |
| TMEM178A | −1.75 | −1.61 |
| TMEM229B | −1.17 | −1.78 |
| TMEM72 | −1.47 | −3.84 |
| TMEM8B | −1.29 | −1.58 |
| TNF | −0.17 | −1.67 |
| TNFRSF13C | −0.97 | −2.02 |
| TPRG1 | −1.70 | −2.14 |
| TREM1 | −1.13 | −3.97 |
| TREML2 | −0.90 | −2.21 |
| TRIM3 | −0.90 | −2.34 |
| TTC8 | −0.68 | −2.33 |
| TTI2 | −0.97 | −2.06 |
| TXNIP | −1.41 | −1.41 |
| UBASH3B | −1.36 | −2.21 |
| UNC45B | −2.04 | −3.05 |
| USP2 | −0.48 | −2.77 |
| USP2-AS1 | −0.05 | −2.78 |
| USP44 | −0.96 | −2.35 |
| VNN2 | −0.87 | −0.25 |
| WDFY3 | −1.05 | −0.72 |
| XCL1 | −2.11 | −2.69 |
| XCL2 | −1.70 | −2.57 |
| XCR1 | −2.09 | −3.43 |
| ZMYND12 | −1.16 | −7.81 |
| ZNF14 | 0.02 | −1.73 |
| ZNF391 | −1.38 | −3.33 |
| ZNF516 | −1.48 | −2.50 |
| ZNF608 | −1.01 | −0.53 |

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A CD16+CD57+NK-92MI cell that natively expresses CD16 and CD57 and that exhibits IL-2 independent growth.

2. The cell of claim 1, wherein NKG2D is expressed on a cell surface of the cell in higher quantities as compared to NK-92MI cells, and/or wherein CD3 is not present on a surface of the cell and CD56 is present on the surface of the cell.

3. The cell of claim 1, wherein CD4+, CD25+, NKp30+, NKp44+, NKp46+, CD27+, OX40+, CD107a+, NKG2A+, PD-1+, TIGIT+, and/or CD158+is/are present on a surface of the cell.

4. The cell of claim 1, wherein the cell has enhanced direct cytotoxicity against Ramos cells as compared to NK-92MI cells, and/or wherein the cell has ADCC in the presence of a target cell and an antibody against a surface protein on the target cell.

5. The cell of claim 1, wherein the cell has, post-thaw and expansion, maintained high expression of CD57, CD16, and NKG2D as compared to NK-92MI cells.

6. The cell of claim 1, wherein the cell has a faster replenishment post degranulation, and/or a faster cell doubling time as compared to NK-92MI cells.

7. The cell of claim 1, wherein the cell is transfected with a recombinant nucleic acid that encodes a chimeric antigen receptor, a homing receptor, a chemokine receptor, a TGF-β trap, and/or a checkpoint inhibitor.

8. A cell culture comprising a plurality of dividing cells in a culture medium, wherein the cells are CD16+CD57+NK-92MI cells that natively express CD16 and CD57 and that exhibit IL-2 independent growth, and wherein the medium is substantially free of IL-2.

9. The cell culture of claim 8, wherein the plurality of dividing cells are maintained in a single culture container from a start of the culture and during growth until a predetermined quantity of cells is obtained.

10. The cell culture of claim 8, wherein the culture contains at least 1×10⁷ cells per culture container.

11. The cell culture of claim 8, wherein the medium contains AB serum.

12. A method of preparing CD16+CD57+NK-92MI cells that natively express CD16 and CD57 and that exhibit IL-2 independent growth, comprising:
providing a plurality of NK-92MI cells, and
using anti-CD16 antibodies and anti-CD57 antibodies to enrich the CD16+CD57+NK-92MI cells that natively express CD16 and CD57.

13. The method of claim 12, wherein the each of the anti-CD16 antibodies and anti-CD57 antibodies are fluorescence-labeled and wherein the step of using the antibodies comprises fluorescence activated cell sorting.

14. The method of claim 13, wherein the fluorescence activated cell sorting is sequentially performed with respect to use of the anti-CD16 antibodies and anti-CD57 antibodies.

15. The method of claim 13, wherein the fluorescence activated cell sorting comprises iterative rounds of fluorescence activated cell sorting.

16. A method of treating a cancer, comprising:
administering to an individual in need thereof a composition comprising a medium that contains a therapeutically effective quantity of cells according to claim 1.

17. The method of claim 16, wherein the individual is a mammal, and/or wherein the cancer is a solid tumor.

18. The method of claim 16, wherein the medium is a growth medium, a cryopreservation medium, or a pharmaceutically acceptable medium for infusion, and wherein the NK-92MI derived cells are optionally irradiated.

19. The method of claim 16, wherein the cell is transfected with a recombinant nucleic acid that encodes a chimeric antigen receptor, a homing receptor, a chemokine receptor, a TGF-β trap, and/or a checkpoint inhibitor.

20. The method of claim 16, further comprising a step of co-administering an antibody, a checkpoint inhibitor, an immune stimulant, a cancer vaccine, and/or metronomic low-dose chemotherapy.
